(19) United States
(12) Patent Application Publication
(10) Pub. No.: US 2024/0252457 A1
(43) Pub. Date: Aug. 1, 2024

(54) Treatment or Prevention of Ischaemia Reperfusion Injury

(71) Applicant: Cambridge Enterprise Limited, Cambridge, Cambridgeshire (GB)

(72) Inventors: Thomas Krieg, Cambridge (GB); Michael Patrick Murphy, Cambridge (GB); Hiran A. Prag, Cambridge (GB)

(21) Appl. No.: 18/568,243

(22) PCT Filed: Jun. 8, 2022
(86) PCT No.: PCT/GB2022/051436

§ 371 Date: Dec. 7, 2023

(30) Foreign Application Priority Data
Jun. 16, 2021 (GB) 2108594.9

(51) Int. Cl. A61K 31/194; A61K 9/00; A61K 45/06; A61P 9/10

(52) U.S. Cl. CPC A61K 31/194; A61K 9/0019; A61K 45/06; A61P 9/10

(57) ABSTRACT

The present invention relates to compositions for use in treating or preventing ischaemia reperfusion injury. In particular, the present invention relates to a composition comprising a salt of formula (I): for use in treating or preventing ischaemia reperfusion injury in a subject; wherein said composition has a pH of from about 4.0 to about 7.0. and wherein X, Y, n, m, Z, A and B are as defined herein.

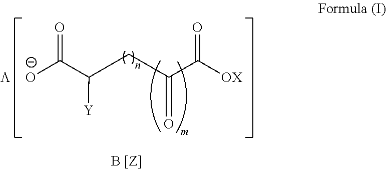

### FIELD

The present invention relates to compositions for use in the treatment or prevention of ischaemia reperfusion (IR) injury.

### BACKGROUND TO THE INVENTION

Ischaemia reperfusion injuries are major health problems worldwide. Reperfusion injuries following myocardial infarction, ischaemic strokes, elective surgery, resuscitation after cessation of cardiac function, local ischaemia and reperfusion injury due to injury or medical intervention, and organ storage and transplantation are prominent causes of such injuries.

Recent estimations (Feigin V L et al. Global and regional burden of stroke during 1990-2010: findings from the Global Burden of Disease Study 2010. Lancet. 2014; 383(9913):245-254) indicate that the global prevalence rate is 5 strokes per 1000 person-years, corresponding to 33 million people living after a stroke. 5.9 million people suffered a stroke-related death in 2010, and stroke resulted in more than 102 million lost disability-adjusted life years (DALYs), corresponding to the sum of premature death and years of healthy life lost attributed to disability.

In recent decades, little progress has been made in stroke therapy options and, correspondingly, little progress has been made in reducing mortality/morbidity rates.

Currently, there is no specific therapy for ischaemic stroke other than stopping the ischaemic insult via thrombectomy or thrombolysis. Minimising the ischaemic time is key to salvaging ischaemic tissue.

Once the ischaemic insult is stopped, the reperfusion of blood into ischaemic tissue is itself damaging, resulting in stroke ischaemia/reperfusion (IR) injury.

Myocardial infarction (MI) and the consequential failure of the heart as a pump is a leading cause of mortality in the developed world (Davidson S M et al. Multitarget Strategies to Reduce Myocardial Ischemia/Reperfusion Injury. Journal of the American College of Cardiology. 2019; 73:89-99). Early reperfusion of the ischemic myocardium by primary percutaneous coronary intervention (PPCI) is essential to prevent cardiomyocyte death and salvage the heart (Murphy E et al. Mechanisms underlying acute protection from cardiac ischemia-reperfusion injury. Physiological Reviews. 2008; 88:581-609). However, the reintroduction of oxygen into the cardiac tissue accelerates cardiomyocyte death via ischaemia/reperfusion (IR) injury (Kalogeris T et al. Cell biology of ischemia/reperfusion injury. International Review of Cell and Molecular Biology. 2012; 298:229-317). While an increasing number of patients survive their initial infarct, IR injury is a major driver of post-MI heart failure (Kloner R A et al. New and revisited approaches to preserving the reperfused myocardium. Nature Reviews Cardiology. 2017; 14:679-693).

Ischaemia reperfusion injury is also prevalent during organ transplantation, because an organ to be transplanted will be exposed to numerous and often extended periods of global warm and cold ischaemia, before implantation into the recipient (Martin J L et al. Succinate accumulation drives ischaemia-reperfusion injury during organ transplantation. Nature Metabolism. 2019; 1:966-974). As current legislation in numerous countries prevents the administration of drugs to the donor, there is limited opportunity to intervene pharmacologically to minimise damage to the organ to be implanted.

Despite the clear detrimental effects of ischaemia reperfusion injury, pharmacological interventions to reduce IR injury remain underdeveloped.

Historically, IR injury was thought of as a random and chaotic series of damaging events resulting in the formation of reactive oxygen species (ROS) from reperfusing ischaemic tissue.

It has recently been shown that metabolic mechanisms within mitochondria are central to IR injury. The citric acid cycle metabolite succinate has been found to accumulate in tissue during ischaemia. During reperfusion, this succinate is rapidly oxidised by succinate dehydrogenase (SDH), driving a spike in ROS production by the mitochondrial complex I². This ROS pulse, together with calcium dysregulation and ATP depletion, initiates a cascade of damaging events culminating in reperfusion injury. This reperfusion leads to damage over and above that of the ischaemia alone. Developing drugs that reduce reperfusion injury following ischaemia, such as caused by ischaemic stroke or myocardial infarction, thus provides a therapeutic window to reduce organ damage.

SDH is a key enzyme in succinate formation during ischaemia and its oxidation upon reperfusion. It is thought that ischaemia reperfusion injury may be ameliorated by altering succinate metabolism, either by preventing its accumulation during ischaemia (thus less succinate is available to be oxidised during reperfusion) or directly blocking its oxidation during reperfusion (e.g. by inhibiting SDH).

As set out in WO 2016/001686, dimethyl malonate (DMM) has previously been shown to be protective when administered prior to and throughout ischaemia. However, as shown in FIG. 8, DMM was found not to be protective when infused at or immediately prior to reperfusion, as malonate was released too slowly.

Tuned malonate prodrugs have been found to be protective when administered at the clinically relevant point of reperfusion, where they rapidly diffuse into the ischaemic tissue and there hydrolyse to release malonate (Prag H A et al., Prodrugs of Malonate with Enhanced Intracellular Delivery Protect Against Cardiac Ischemia-Reperfusion Injury In Vivo). Surprisingly, malonate itself (administered as the disodium salt, disodium malonate (DSM)) is also protective against myocardial IR injury in small animal ex vivo and large animal models (Falls-Lacalle L et al. Succinate dehydrogenase inhibition with malonate during reperfusion reduces infarct size by preventing mitochondrial permeability transition. Cardiovascular Research. 2016; 109:374-384). This cardioprotection by DSM was unexpected as malonate is a dicarboxylate and carries two negative charges at physiological pH (i.e. at approximately pH 7.4) in the blood, which will impede its uptake across biological membranes. However, high concentrations and extended incubation times are required, despite a large pool of excess malonate, to see malonate-dependent effects in vitro. The mechanism of uptake and action of DSM in the treatment of IR injuries, and thus the potential for translation to the clinic, was previously unclear (Kula-Alwar D et al. Targeting Succinate Metabolism in Ischemia/Reperfusion Injury. Circulation. 2019 Dec. 10; 140(24):1968-1970).

Thus, there remain clear unmet clinical needs for pharmaceutical compositions which further reduce the extent of IR injury following reperfusion of ischaemic tissue, for pharmaceutical compositions which are able to selectively target ischaemic tissue, and for pharmaceutical compositions which are more rapidly taken up by ischaemic tissue.

### SUMMARY OF THE INVENTION

In view of the above, there remains a need to develop improved compositions for use in the treatment and prevention of ischaemia reperfusion injury.

Ideally, such compounds and compositions could also be administered in conjunction with therapies intended to chemically or mechanically remove an ischaemic event, e.g. a blood clot.

The present invention arises from the surprising finding that the uptake of certain salts, including malonate salts, into cells is dramatically enhanced at a lowered pH. It has been discovered that driving the uptake of malonate with lowered pH substantially blunted mitochondrial oxygen consumption in cells, which slows the oxidation of succinate during the critical first minutes of reperfusion, where the conditions are optimal for the initiation of damage. This finding thus provides an opportunity dramatically improved treatments for ischaemia reperfusion injuries.

The intracellular delivery of malonate is greatly enhanced in cells at a lower (more acidic) pH (FIG. 1). The increase in malonate uptake at pH 6 compared to pH 7.4 is roughly 15-fold. Thus, acidic pH dramatically enhances malonate's ability to cross the plasma membrane and enter cells. Furthermore, in the ex vivo Langendorff perfused heart, malonate uptake is also greatly enhanced when infused at a low pH compared to when infused at more neutral conditions (FIG. 2). To confirm that the pH is important in driving the uptake of malonate across the membrane, we compared the uptake of malonate in cells at different pH to a modified version of malonate, 3-amino-3-oxypropanoic acid (3A3OPA). 3A3OPA was unable to react in the same manner as malonate to a lowered pH, with similar levels of uptake achieved for 3A3OPA at both low and neutral pH (FIG. 5). Overall the data presented here underpins the discovery that artificially lowering the pH of an ischaemic tissue dramatically enhances the intracellular delivery of malonate. This selective uptake of malonate by ischaemic tissue greatly increases the concentration of malonate accumulating in the tissue, which in turn reduces the extent of IR injury caused upon reperfusion.

The present invention relates in particular to a composition comprising a salt of formula (I):

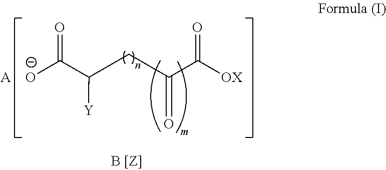

for use in treating or preventing ischaemia reperfusion injury in a subject;
wherein X is selected from a negative charge, H, or C₁-C₁₂ alkyl;
wherein Y is selected from H, OH or C₁-C₁₂ alkyl;
wherein n is 0 or 1;
wherein m is 0 or 1;
wherein Z is one or more pharmaceutically acceptable cations; and
wherein A and B are independently any integer, such that the net charge of the salt is 0, and wherein said composition has a pH of from about 4.0 to about 7.0.

Preferably, the above salt of formula (I) is a salt of formula (II) as defined below, more preferably a malonate salt.

The present invention also relates to a combination therapy for use in treating or preventing ischaemia reperfusion injury in a subject, said combination therapy comprising (1) a pH lowering component having a pH of from about 4.0 to about 7.0 and (2) a salt of formula (I) as defined herein.

The present also relates to a unit dosage form comprising a composition as defined above, wherein the total volume of the unit dosage form is less than about 20 ml. Such unit dosage forms are particularly useful when administered as part of a thrombectomy treatment proximal or distal to the obstructing clot and directly to ischaemic tissue.

### BRIEF DESCRIPTION OF THE DRAWINGS

FIG. 1 demonstrates the pH-dependence of malonate uptake in C2C12 myoblast cells. C2C12 cells were incubated with malonate at pH 8, 7.4 or 6 and the levels within cells measured by LC-MS/MS (mean±/−S.E.M., n=4).

FIG. 2 demonstrates pH-dependence of malonate uptake in the murine Langendorff perfused heart. Malonate (5 mM) was infused into Langendorff perfused mouse hearts at either pH 7.4 or 6. The heart flushed to remove vessel malonate and malonate levels measured by LC-MS/MS (mean±/−S.E.M., n=4).

FIG. 3 demonstrates ischaemia-dependent uptake of malonate in the murine Langendorff perfused heart. Langendorff mouse hearts were reperfused with malonate (5 mM) after either 0, 5, 10, or 20 min ischaemia). The heart flushed to remove vessel malonate and malonate levels measured by LC-MS/MS (mean±/−S.E.M., n=4-5).

FIG. 4 demonstrates the effect of malonate uptake during ischaemia on cardioprotection in vivo. Mice were subjected to left anterior descending coronary artery ligation heart attack model (30 min ischaemia, 2 hr reperfusion) and malonate infused either before the onset of ischaemia or during initial reperfusion. Infarct size was determined by staining the heart with triphenyltetrazolium chloride (mean±/−S.E.M., n=5-8).

FIG. 5 demonstrates the fold-enhancement update of malonate by C2C12 cells. C2C12 cells were treated with either malonate or 3-amino-3-oxypropanoic acid at pH 7.4 or 6 and their intracellular levels measured by LC-MS/MS (mean±/−S.E.M., n=6).

FIG. 6 shows the effect of a representative compound, disodium malonate, on infarct volume following ischaemic stroke vs a saline control.

FIG. 7 shows the effect of a representative compound, disodium malonate, on cerebral blood flow (CBF) at various points during ischaemic stroke and reperfusion, vs a saline control.

FIG. 8 shows the effect of a comparative compound, dimethyl malonate, on infarct volume following ischaemic stroke vs a saline control.

FIG. 9 shows in vitro uptake of a representative compound, disodium malonate, by tissues and cells.

FIG. 10 shows in vivo uptake of a representative compound, disodium malonate, by mouse tissues.

FIG. 11 shows a mouse model of acute ischaemic stroke in vivo.

FIG. 12 shows a MALDI image of succinate levels in acute middle cerebral artery (MCA) occlusion.

FIG. 13 shows accumulation of succinate during stroke in mouse brains.

FIG. 14 shows accumulation of succinate during stroke in human brains.

FIG. 15 shows ischaemic succinate levels in brain tissue from a mouse stroke model.

FIG. 16 shows Complex I activity in brain tissue from a mouse stroke model.

FIG. 17 shows levels of malonate in mouse brain tissue following administration of disodium malonate from 5 minutes before until 5 minutes after reperfusion.

FIG. 18 shows levels of malonate in mouse cerebrospinal fluid (CSF) following administration of disodium malonate from 5 minutes before until 5 minutes after reperfusion.

FIG. 19 shows the effect of disodium malonate on infarct size in an acute ischaemic mouse stroke model.

FIG. 20 shows the brains used to generate data presented in FIG. 14. Pale brain regions represent infarct regions.

FIG. 21 shows plasma succinate levels in venous blood from patients undergoing thrombolysis due to an acute ischemic stroke.

FIG. 22 shows the pH-dependence of malonate uptake in H9c2 myoblasts.

FIG. 23 shows succinate levels in H9c2 myoblasts following administration of malonate at different pH values.

FIG. 24 shows succinate levels in C2C12 cells incubated with DSM (5 mM) for 15 min at a range of pH values.

FIG. 25 shows lactate levels in the Langendorff heart when measured after 20 min ischemia and 1 min reperfusion with and without 5 mM DSM.

FIG. 26 shows infarct size in murine LAD MI model with 100 μl bolus of 8 mg/kg DSM, pH 4 acid control or 8 mg/kg pH 4 formulated malonate measured at reperfusion after 30 min ischemia.

FIG. 27 shows malonate levels in murine Langendorff hearts perfused at pH 6 for 5 min with 5 mM DSM±lactate (50 mM; Lac) or MCT1 inhibitor.

FIG. 28 shows infarct size in murine LAD model with infusion of vehicle (ethanol/Cremphor EL in saline)±cyclosporin A (10 mg/kg) or DSM (160 mg/kg) at reperfusion.

### DETAILED DESCRIPTION OF THE INVENTION

In particular, the invention relates to compositions comprising a salt of formula (I):

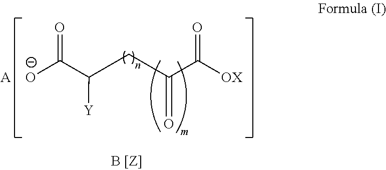

for use in treating or preventing ischaemia reperfusion injury in a subject, wherein said composition has a pH of from about 4.0 to about 7.0.

In the salt of formula (I), X is selected from a negative charge, H, or C₁-C₁₂ alkyl group. Preferably, X is selected from a negative charge, H or C₁-C₁₀ alkyl. More preferably, X is selected from a negative charge, H or C₁-C₈ alkyl.

In the salt of formula (I), Y is selected from H, OH or C₁-C₁₂ alkyl. Preferably, Y is selected from H, OH or C₁-C₁₀ alkyl. More preferably, Y is selected from H, OH or C₁-C₅ alkyl.

In the salt of formula (I), n is 0 or 1.

In the salt of formula (I) above, m is 0 or 1.

In the salt of formula (I) above, Z is one or more pharmaceutically acceptable cations. Each Z is a pharmaceutically acceptable cation, provided that the net charge of the salt of formula (I) is 0. For example, Z may comprise a cation with a +1 charge, a +2, or a +3 charge. Preferably, Z comprises: a cation selected from Li⁺, Na⁺, K⁺, Rb⁺, Cs⁺, Mg²⁺, Ca²⁺, Sr²⁺, Ba²⁺, Cu⁺, Cu²⁺, Fe²⁺, Fe³⁺, Pb²⁺, Ni²⁺, Ag⁺, Sn²⁺, Cr³⁺, Zn²⁺, Al³⁺, NH₄⁺ and/or triphenylphosphonium; or a cation selected from Li⁺, Na⁺, K⁺, Rb⁺, Cs⁺, Mg²⁺, Ca²⁺, Sr²⁺, Ba²⁺, Pb²⁺, Ni²⁺, Ag⁺, Sn²⁺, Cr³⁺, Zn²⁺, Al³⁺, NH₄⁺ and/or triphenylphosphonium; or a cation selected from Li⁺, Na⁺, K⁺, Rb⁺, Cs⁺, Mg²⁺, Ca²⁺, Sr²⁺, Ba²⁺, Pb²⁺, Ni²⁺, Sn²⁺, Cr³⁺, Zn²⁺, NH₄⁺ and/or alkyl triphenylphosphonium; or a cation selected from Li⁺, Na⁺, K⁺, Mg²⁺, Ca²⁺, Zn²⁺, NH₄⁺ and/or triphenylphosphonium.

When the cation is a triphenylphosphonium cation, it is preferably a C₁-C₁₂ alkyl triphenylphosphonium cation, more preferably decyl(triphenyl)phosphonium cation.

In the salt of formula (I), A and B may independently be any integer (for example, independently 1, 2 or 3), provided that the net charge on the salt of formula (I) is 0. For example, when Z is a +3 cation and the anion is a −2 anion, A is 3 and Bis 2, such that the net charge on the salt is 0. In one embodiment, A and B are each independently selected from 1 and 2; for example A is 1 and B is 1 or 2.

In the salt of formula (I), Z may be a single ion, for example a Nation, or multiple ions, such as a Na⁺ and a K⁺ ion. Thus, salts with multiple cations (such as sodium potassium malonate) are encompassed by formula (I).

In a preferred embodiment of the composition for use according to the invention, the salt of formula (I) has a value of n of 0, and a value of m of 0. In this case, the salt is a salt of formula (II):

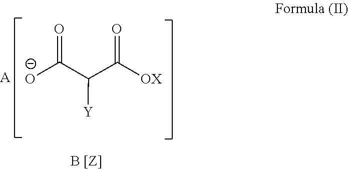

wherein A, B, Z, Y and X are as defined above.

The salt of formula (I) may preferably be a malonate salt. In this case, the salt is of formula (II) and Y is a H atom.

For example, the salt of formula (II) may be disodium malonate, monosodium malonate, dipotassium malonate, monopotassium malonate, dilithium malonate, monolithium malonate, calcium malonate, magnesium malonate, ammonium malonate, aluminium malonate or zinc malonate. Optionally, the salt of formula (II) is disodium malonate.

The salt of formula (I) may also be a salt of formula (II), wherein Y is a C₁-C₅ alkyl, more preferably a butyl group.

The composition for use according to the present invention has a pH of less than about 7. Preferably, the composition has a pH of less than about 6.9, preferably less than about 6.8, preferably less than about 6.7, preferably less than about 6.6, and preferably less than about 6.5.

The composition for use according to the present invention has a pH of greater than about 4. Preferably, the composition has a pH of greater than about 4.3, preferably greater than about 4.5, preferably greater than about 4.8, preferably greater than about 5, preferably greater than about 5.3, and preferably greater than about 5.5.

Preferably, the composition for use according to the invention has a pH of from about 4.1 to about 6.9, preferably from about 4.4 to about 6.8, preferably from about 4.7 to about 6.7, preferably from about 5 to about 6.6, and preferably from about 5.3 to about 6.6.

The composition for use according to the invention may comprise one or more buffering agents which maintain the pH of the composition within a particular pH range as set out above. Any buffering agent which is able to maintain pH within a range as set out above may be used. For example, the composition of the present invention may comprise as a buffering agent one or more of: citric acid, acetic acid, lactic acid, gluconic acid, aspartic acid, glutamic acid, tartaric acid, succinic acid, malic acid, fumaric acid, carbonic acid, a carbonate salt, a bicarbonate salt, or α-Ketoglutaric acid.

The composition for use according to the invention may be administered in combination with one or more blood thinning agents and/or one or more lysis agent and/or one or more antiplatelet drugs intended to stop the ischaemic insult via thrombolysis. For example, the composition may be administered in combination with: one or more blood thinning agents selected from (but not limited to) Coumadin™ (warfarin); Pradaxa™ (dabigatran); Xarelto™ (rivaroxaban) and Eliquis™ (apixaban), Fondaparinux, unfractionated heparin, low molecular weight heparins including but not limited to enoxaparin and deltaparin; one or more thrombolytic agents selected from (but not limited to Streptokinase (SK), Urokinase, Lanoteplase, Reteplase, Staphylokinase, Tenecteplase and Alteplase; and/or one or more antiplatelet drugs selected from (but not limited to) aspirin, clopidogrel or ticagrelor.

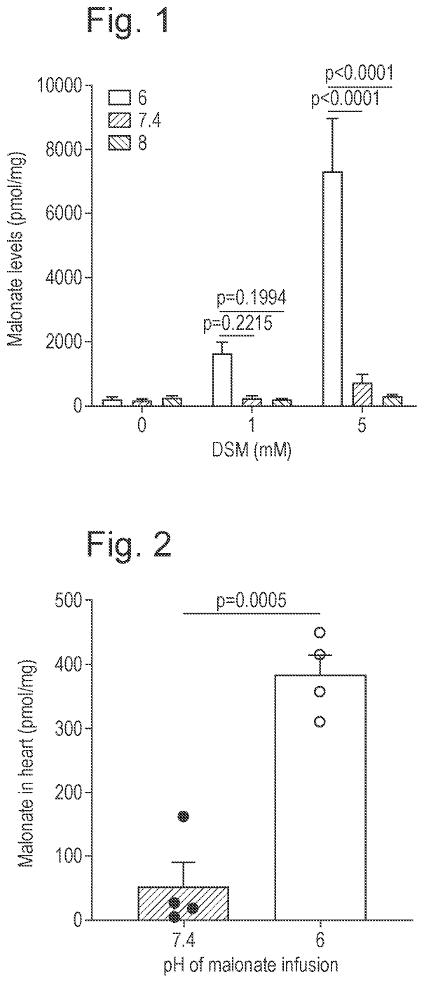

The use of compositions of the present invention nullifies a number of barriers to clinical translation. The active compounds of the compositions of the present invention, such as malonate salts, can enter mitochondria by endogenous transport mechanisms, thus allowing the compound to reach the target site in a timely manner. As discussed above and as demonstrated in FIGS. 1 and 2, the rate of uptake of such salts to ischaemic tissue is advantageously increased as a result of the low pH of the compositions administered to said ischaemic tissue. This results in a selective uptake of the salt of formula (I) to the ischaemic tissue. Thus, a high effective concentration of the salt of formula (I) is able to accumulate in the tissue, where it is able to minimise succinate oxidation and tissue damage in the ischaemic tissue upon reperfusion of the tissue.

Furthermore, the active compounds of the present invention, and in particular malonate salts, have limited toxicity, well established metabolism and have been used as excipients in pharmaceutical development.

As used herein, reperfusion refers to the point at which blood flow into the ischaemic tissue begins following the ischaemic event. Reperfusion may occur naturally (such as following a transient ischaemic attack), or may be artificially initiated, for example in a medical setting. When reperfusion is initiated, it may by initiated by any method known in the art capable of removing a blockage in blood flow, such as by either mechanical or chemical means. Preferably, reperfusion is initiated by thrombolysis (for example, by administering blood thinning agents or application of lysis agents to the patient), and/or by thrombectomy. When reperfusion is initiated, this may be in combination with administering the composition of the invention. For example, the initiation of reperfusion may occur before the start of administration of the composition of the invention. Alternatively, initiation of reperfusion may occur at approximately the same time as the start of administration of the composition of the invention—for example when a lysis agent is administered as part of the composition of the invention as set out above. Alternatively, initiation of reperfusion may occur after the start of administration of the composition of the invention.

Suitable blood thinning agents may be selected from Coumadin™ (warfarin); Pradaxa™ (dabigatran); Xarelto™ (rivaroxaban) and Eliquis™ (apixaban), Fondaparinux, unfractionated heparin, low molecular weight heparins including but not limited to enoxaparin and deltaparin, thrombolytic agents including but not limited to Streptokinase (SK), Urokinase, Lanoteplase, Reteplase, Staphylokinase, Tenecteplase and Alteplase, or antiplatelet drugs such as aspirin, clopidogrel or ticagrelor.

The composition for use according to the invention may be administered at any point following suspected ischaemia. For example, when it is suspected that ischaemic stroke has occurred, the composition may be administered following observance of one or more stroke symptoms selected from: sudden numbness or weakness in the face, arm, or leg, sudden confusion, trouble speaking, or difficulty understanding speech, sudden trouble seeing in one or both eyes, sudden trouble walking, dizziness, loss of balance, or lack of coordination, sudden severe headache, complete paralysis of one side of the body, difficulty swallowing (dysphagia) and loss of consciousness. When it is suspected that myocardial infarction (MI) has occurred, the composition may be administered following observance of one or more symptoms such as chest pressure or pain, light-headedness or dizziness, sweating, shortness of breath, nausea or vomiting, anxiety and coughing or wheezing.

An advantage of the present invention is that the composition for use according to the invention is beneficial for the treatment and prevention of ischaemic stroke reperfusion injury, but is not harmful in the case of haemorrhagic stroke. Thus, the composition of the invention can be administered to a patient suspected of having a stroke before diagnosis of the type of stroke. As such, the composition may be used as an emergency medication before diagnosis of type of stroke, which minimises the damage caused upon reperfusion. In the case of administration as an emergency medication (such as administration by emergency responders, for example paramedics), the composition of the invention is preferably administered to a patient suspected of suffering a stroke at any point before diagnosis of the type of stroke, and preferably within about 4 hours of the onset of stroke symptoms, preferably within about 3 hours of the onset of stroke symptoms, preferably within about 2 hours of the onset of stroke symptoms and more preferably within about 1 hour of the onset of stroke symptoms. The composition of the invention may be administered in a setting where it is possible to initiate treatment extremely quickly after stroke symptoms begin, for example in a ward on a hospital. Initiation of administration of the composition according to the invention can thus begin within about 50 minutes of onset of stroke symptoms, preferably within about 40 minutes of onset of stroke symptoms, preferably within about 30 minutes of onset of stroke symptoms, preferably within about 20 minutes of onset of stroke symptoms and most preferably within about 15 minutes of onset of stroke symptoms.

As discussed above, ischaemia during organ transplantation has numerous deleterious effects on the outcome of the surgery. An organ transplant recipient may be treated with a composition according to the present invention prior to implantation of the donated organ. Then, when the organ is reperfused, there is a reservoir of malonate which is able to minimise succinate oxidation and tissue damage in the implanted organ following reperfusion. Alternatively or additionally, the composition of the present invention may be administered to the transplanted organ itself prior to or at the point of reperfusion. This administration may be in vivo i.e. once the organ has been implanted into the recipient, or ex vivo. Thus, the present invention also relates to a method of administering a composition as defined herein to an organ ex vivo. In both cases, this reduces succinate oxidation and reperfusion damage to the transplanted organ following reperfusion. Therefore, the present invention has substantial benefits to clinical practice during organ transplantation.

The present invention is applicable to any clinical situation where reperfusion may occur following ischaemia. In addition to heart attack, ischaemic stroke and ischaemia that occurs during organ transplant, the compositions for use according to the invention may also be used, for example, in treating kidney IR injury, and treating IR that occurs during elective surgery. The composition for use according to the invention can also be used to treat, for example, reperfusion injuries following elective surgery, resuscitation after cessation of cardiac function, and local ischaemia and IR injury due to injury or medical intervention. For example, the composition for use according to the invention can be used to treat IR injury to the brain following cardiac resuscitation.

The composition of the invention may first be administered to a patient following the onset of ischaemia but before reperfusion. Preferably, administration of the compound of the invention then continues until reperfusion is established, preferably continues during reperfusion, and preferably continues until reperfusion is complete.

Administering the composition of the invention for a period of time before initiation or onset of reperfusion ensures that the pH of the ischaemic tissue is reduced to below normal physiological pH, and that the salt of formula (I) is advantageously able to accumulate in concentrations sufficient to minimise the production of ROS on reperfusion, protecting the ischaemic tissue from IR injury. As such, it may be preferable to first administer the composition of the invention to the patient as soon as possible after onset of ischaemia symptoms. The composition of the invention is thus preferably administered at least on initiation or at the onset of reperfusion, optionally for a time of at least about 5 minutes before initiation or onset of reperfusion, optionally 10 minutes, optionally 15 minutes and optionally 20 minutes or more before initiation or onset of reperfusion.

Alternatively, the composition of the invention may first be administered to a patient following initiation or onset of reperfusion. In this case, the composition of the invention is preferably first administered to the patient as soon as possible following initiation of reperfusion to minimise IR injury. Following initial administration of the composition of the invention to the patient, the administration preferably continues until reperfusion is complete.

Alternatively, the composition of the invention may first be administered to a patient at the point of reperfusion. Preferably, administration of the composition of the invention then continues until reperfusion is complete.

The composition of the invention may be administered to a patient in combination with a treatment used or intended to remove a blockage in blood flow i.e. a treatment used or intended to initiate reperfusion. This may occur simultaneously, for example when the composition is administered to a patient at the point of reperfusion, or separately; for example the composition is administered following ischaemia but before reperfusion, and the treatment to initiate reperfusion is started subsequently (either during the administration of composition of the invention, or after such administration has stopped). Preferably, the composition of the invention is administered to a patient in combination with a thrombolysis treatment and/or thrombectomy. Preferably, the thrombolysis treatment is selected from application of blood thinning agents or application of lysis agents to the patient, for example those agents above.

It is particularly preferred that the composition of the invention be administered to a patient in combination with thrombectomy, i.e. treatment involving removal of a blockage to blood flow, for example a blood clot, using mechanical means. As an example, a thrombectomy catheter may be used to enter an obstructed blood vessel and physically remove an ischaemic insult. Administering the composition for use according to the invention in combination with a thrombectomy treatment allows the composition of the invention to be introduced directly to the site of the ischaemic insult, which allows local pH in the ischaemic tissue to be reduced. Any mechanical means for thrombectomy may be used in combination with the composition for use according to the invention.

When a thrombectomy is performed, the composition for use according to the invention may be administered directly to the occluded vessel via the obstruction by the mechanical means used to remove the clot. In this case, the mechanical means for performing the thrombectomy may comprise a composition according to the invention. For example, the mechanical means for performing the thrombectomy may be coated with a composition according to the invention, which may then be released into an occluded vessel by diffusion. Alternatively, the mechanical means may be configured to release the composition for use according to the invention as a bolus or a unit dosage form as defined herein within the occluded vessel proximal or distal to the obstructing clot. Alternatively, the mechanical means may comprise a catheter through which a composition for use according to the invention may be introduced to the occluded vessel.

Alternatively, when a thrombectomy is performed, the composition for use according to the invention may be introduced to the occluded vessel via a catheter which is separate to the mechanical means used to perform the thrombectomy. For example, the composition of the invention may be introduced to the occluded vessel via a catheter and the catheter withdrawn from the body before the mechanical means for performing the thrombectomy is introduced to the occluded vessel and the thrombectomy performed. Alternatively, the composition of the invention may be introduced to the occluded vessel via a catheter after the thrombectomy has been performed and the mechanical means for performing the thrombectomy has been removed from the occluded vessel.

The composition for use according to the invention may comprise, or be administered as part of a combination therapy with, one or more further components intended to prevent or minimise tissue damage. For example, the composition for use according to the invention may be administered with other agents or interventions for targeting ischaemia reperfusion injury.

The composition for use according to the invention may be administered in combination with an inhibitor of the mitochondrial permeability transition pore (PTP). The inhibitor of mitochondrial PTP may, for example, be cyclosporin A (CsA).

As set out above, a key advantage of the present invention is that administering a composition as defined herein results in a decrease of the pH in the ischaemic tissue. This acidification of the tissue drives the selective uptake of the salt of formula (I), such as malonate salts, by the ischaemic tissue and results in advantageously high concentrations of such salts within the ischaemic tissue. This advantageously fast and selective uptake of the salt of formula (I) reduces the reperfusion damage caused to the ischaemic tissue upon reperfusion.

The composition for use according to the invention may be administered by any method in the art. For example, the composition of the invention may be administered orally, topically, subcutaneously, parenterally, intramuscularly, intraperitoneally, intraocularly, intranasally, intra-arterially or intravenously. Preferably, the composition for use according to the invention is administered intravenously, intra-arterially intraperitoneally, or parenterally, for example directly to a tissue undergoing ischaemia, or suspected of undergoing ischaemia.

The composition for use according to the invention may be in any form suitable for administration to a subject in need thereof, but is preferably in the form of a solution for infusion. Optionally, the solution for infusion comprises one or more buffering agents as defined above. Optionally, the solution for infusion is an isotonic solution. Optionally, the solution for infusion comprises sodium chloride i.e. it is a saline solution. As discussed herein, a mechanical means for thrombectomy may comprise a solution of the composition for use according to the invention, which is released directly into the occluded vessel during thrombectomy. Alternatively, the composition for use according to the invention may be in the form of a solution for infusion intended for intravenous administration.

Alternatively, the composition for use according to the invention may be formulated and administered as a controlled or sustained release composition. For example, the composition for use according to the invention may be formulated in a lipophilic depot (e.g. fatty acids, waxes, oils) or comprise a polymer coating (e.g. poloxamers or poloxamines). When the composition for use according to the invention comprises a polymer coating, the polymer may be one that releases acid on hydrolysis within the body, for example, polylactic acid (PLA) or polyglycolic acid (PGA). This has the advantage of further lowering the pH of the tissue at the point of release of the composition, which may further increase the rate of uptake of the salt of formula (I).

The composition for use according to the invention may formulated and administered as a bolus i.e. as a discrete quantity of the composition intended for administration within a time period of less than about 30 minutes, optionally less than about 20 minutes, optionally less than about 10 minutes, and optionally less than about 5 minutes.

When the composition for use according to the invention is administered as a bolus, the total volume of the bolus may vary depending on the specific type of ischaemia reperfusion injury to be treated. For example, the total volume of the bolus may be from about 1 ml to about 500 ml. The bolus may also be in the form of a unit dosage form or a fluid bag for intravenous infusion as defined herein.

Alternatively, the composition for use according to the invention may be continually administered for a time of up to 6 hours from time of the initial administration. For example, the total time of administration of composition may be greater than about 2 minutes, optionally greater than about 5 minutes, optionally greater than about 10 minutes, optionally greater than about 15 minutes. Optionally, the total time of administration of the composition for use according to the invention is less than about 5 hours, optionally less than about 4 hours, optionally less than about 3 hours, and optionally less than about 2 hours.

As set out above, the composition for use according to the invention comprises a salt of formula (I). The salt of formula (I) may be administered at a dose in the range of from about 0.1 mg/kg to about 500 mg/kg of body weight. Preferably, the salt of formula (I) is administered at a dose of greater than about 0.2 mg/kg of body weight, preferably greater than about 0.3 mg/kg of body weight, preferably greater than about 0.4 mg/kg of body weight, and preferably greater than about 0.5 mg/kg of body weight. Preferably, the salt of formula (I) is administered at a dose of less than about 450 mg/kg of body weight, preferably less than about 400 mg/kg of body weight, preferably less than about 350 mg/kg of body weight, and preferably less than about 300 mg/kg of body weight.

The composition for use according to the invention preferably has a concentration of the salt of formula (I) of from about 0.1% (w/v) to about 5% (w/v), preferably from about 0.2% (w/v) to about 4.5% (w/v), preferably from about 0.3% (w/v) to about 4% (w/v), preferably from about 0.4% (w/v) to about 3.5% (w/v), preferably from about 0.5% (w/v) to about 3% (w/v), preferably from about 0.5% (w/v) to about 2.5% (w/v), and preferably from about 0.5% (w/v) to about 2% (w/v).

The composition for use according to the invention preferably has a concentration of the salt of formula (I) of from about 1 mM and 100 mM.

It is envisaged that the total volume of the composition for use according to the invention that is administered to the patient will vary depending on the nature of the IR injury that is being treated, and on the method of administration. For example, when the composition for use according to the invention is administered into a brain artery during the treatment of stroke ischaemia reperfusion injury, the total volume of the composition administered may be less than about 20 ml. However, when the composition for use according to the invention is administered intravenously, the total volume of the composition administered may be less than about 250 ml.

Thus, the present invention also relates to a fluid bag for intravenous infusion, comprising a composition comprising a salt of formula (I) as defined herein, wherein said composition has a pH of from about 4.0 to about 7.0; and wherein the total volume of the composition in the fluid bag is less than about 250 ml. Said composition may have any of the features of the composition defined herein.

Preferably, the composition in the fluid bag has a volume of less than about 225 ml, preferably less than about 200 ml, preferably less than about 175 ml and preferably less than about 150 ml.

Preferably, the composition in the fluid bag has a volume of greater than about 25 ml, preferably greater than about 50 ml, preferably greater than about 75 ml and preferably greater than about 100 ml.

Preferably, the composition in the fluid bag has a volume of from about 25 ml to about 250 ml, preferably from about 50 ml to about 225 ml, preferably from about 75 ml to about 200 ml and preferably from about 100 ml to about 175 ml.

The composition for use according to the invention may additionally comprise one or more pharmaceutically acceptable excipients, carriers or diluents.

Suitable excipients, carriers and diluents can be found in standard pharmaceutical texts. See, for example, Handbook for Pharmaceutical Additives, 3rd Edition (eds. M. Ash and I. Ash), 2007 (Synapse Information Resources, Inc., Endicott, New York, USA) and Remington: The Science and Practice of Pharmacy, 2ist Edition (ed. D. B. Troy) 2006 (Lippincott, Williams and Wilkins, Philadelphia, USA).

Excipients for use in the compositions of the invention include, but are not limited to microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavouring agents, colouring matter or dyes, and, if so desired, emulsifying and/or suspending agents as well, together with such diluents as water, ethanol, propylene glycol, glycerin and various like combinations thereof.

Pharmaceutical carriers include solid diluents or fillers, sterile aqueous media and various non-toxic organic solvents, and the like.

Pharmaceutically acceptable carriers include gums, starches, sugars, cellulosic materials, and mixtures thereof. The compound can be administered to a subject by, for example, subcutaneous implantation of a pellet. The preparation can also be administered by intravenous, intra-arterial, or intramuscular injection of a liquid preparation oral administration of a liquid or solid preparation, or by topical application. Administration can also be accomplished by use of a rectal suppository or a urethral suppository.

Further, as used herein “pharmaceutically acceptable carriers” are well known to those skilled in the art and include, but are not limited to, 0.01-0.1 M and preferably 0.05 M phosphate buffer or 0.9% saline. Additionally, such pharmaceutically acceptable carriers maybe aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Pharmaceutically acceptable parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Pharmaceutically acceptable carriers for controlled or sustained release compositions administrable according to the invention include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Pharmaceutically acceptable carriers include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski and Davis, Soluble Polymer-Enzyme Adducts, Enzymes as Drugs, Hocenberg and Roberts, eds., Wiley-Interscience, New York, N.Y., (1981), pp 367-383). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In a further aspect of the invention there is provided a combination therapy for use in treating or preventing ischaemia reperfusion injury in a subject, comprising (1) a pH lowering component and (2) a salt of formula (I) as defined herein.

The pH lowering component may be administered to the subject, preferably to the ischaemic tissue, either at the same time as or separately from the salt of formula (I).

When the pH lowering component is administered to the subject separately from the salt of formula (I), the pH lowering component may be administered either before or after the salt of formula (I) is administered to the subject. In this case, preferably, the pH lowering component is administered to the subject before the salt of formula (I) is administered to the subject. This ensures that the ischaemic tissue is acidic prior to administering the salt of the present invention, which facilitates rapid uptake of the salt of formula (I) by the ischaemic tissue when the salt of formula (I) is administered. Preferably, the salt of formula (I) is administered to the subject shortly after administering the pH lowering component, for example within 10 minutes of administering the pH lowering component, preferably within about 8 minutes, preferably within about 6 minutes, preferably within about 5 minutes, preferably within about 4 minutes and preferably within about 3 minutes of administering the pH lowering component.

Alternatively, the pH lowering component may be administered to the subject, preferably to the ischaemic tissue, at the same time as administering the salt of formula (I).

The pH lowering component preferably has a pH of less than about 7. Preferably, the pH lowering component has a pH of less than about 6.9, preferably less than about 6.8, preferably less than about 6.7, preferably less than about 6.6, and preferably less than about 6.5.

The pH lowering component preferably has a pH of greater than about 4. Preferably, the pH lowering component has a pH of greater than about 4.3, preferably greater than about 4.5, preferably greater than about 4.8, preferably greater than about 5, preferably greater than about 5.3, and preferably greater than about 5.5.

Preferably, the pH lowering component has a pH of from about 4.1 to about 6.9, preferably from about 4.4 to about 6.8, preferably from about 4.7 to about 6.7, preferably from about 5 to about 6.6, and preferably from about 5.3 to about 6.6.

The pH lowering component may comprise one or more buffering agents which maintain the pH of the pH lowering component within a particular pH range as set out above. Any buffering agent which is able to maintain pH within a range as set out above may be used. For example, the pH lowering component may comprise as a buffering agent one or more of: citric acid, acetic acid, lactic acid, gluconic acid, aspartic acid, glutamic acid, tartaric acid, succinic acid, malic acid, fumaric acid, carbonic acid, a carbonate salt, a bicarbonate salt, or α-Ketoglutaric acid.

The combination therapy for use according to the invention may further comprise one or more further pharmaceutically acceptable components, including any of the further components defined above in relation to the composition of the invention. These optional further components include lysis agents, blood thinning agents, pharmaceutically acceptable excipients, carriers, or diluents, amongst others.

The combination therapy for use according to the invention may further comprise an inhibitor of mitochondrial permeability transition pore (PTP), for example cyclosporin A (CsA).

The combination therapy for use according to the invention may be administered to a subject in need thereof according to any method known in the art, including those methods defined above in relation to administration of the composition of the invention, including administration in combination with a thrombectomy treatment as discussed herein.

The present also relates to a unit dosage form comprising a composition as defined above, wherein the total volume of the unit dosage form is less than about 20 ml. The unit dosage form of the invention may be administered directly to ischaemic tissue, for example by administering the unit dosage form directly into an occluded blood vessel proximal or distal to the obstructing clot. Administering the unit dosage form courses a localised decrease in pH, driving selective uptake of the salt of formula (I) to the ischaemic tissue.

The unit dosage form preferably has a volume of less than about 18 ml, preferably less than about 16 ml, preferably less than about 14 ml, preferably less than about 12 ml and preferably less than about 10 ml.

The unit dosage form preferably has a volume of greater than about 1 ml, preferably greater than about 2 ml, preferably greater than about 3 ml, preferably greater than about 4 ml and preferably greater than about 5 ml.

The unit dosage form preferably has a volume of from about 1 ml to about 18 ml, preferably from about 2 ml to about 16 ml, preferably from about 3 ml to about 14 ml, preferably from about 4 ml to about 12 ml and preferably from about 5 ml to about 10 ml.

The unit dosage form preferably has a concentration of the salt of formula (I) of from about 0.1% (w/v) to about 5% (w/v), preferably from about 0.2% (w/v) to about 4.5% (w/v), preferably from about 0.3% (w/v) to about 4% (w/v), preferably from about 0.4% (w/v) to about 3.5% (w/v), preferably from about 0.5% (w/v) to about 3% (w/v), preferably from about 0.5% (w/v) to about 2.5% (w/v), and preferably from about 0.5% (w/v) to about 2% (w/v).

The unit dosage form preferably has a concentration of the salt of formula (I) of from about 1 mM and 100 mM.

Such unit dosage forms are particularly advantageous when administered proximal or distal to the obstructing clot directly to ischaemic tissue as part of a thrombectomy treatment. The unit dosage form may be administered in combination with a thrombectomy treatment in the same way as discussed above in relation to compositions for use according to the invention.

The present invention also relates to a kit comprising (1) a thrombectomy device and (2) a unit dosage form as defined herein or a a composition comprising a salt of formula (I) as defined herein.

The present invention also relates to a method of treating or preventing ischaemia reperfusion injury in a subject, the method comprising administering a composition or a combination therapy as defined herein to the subject.

The present invention also relates to the use of a composition as defined herein for the manufacture of a medicament for treating or preventing ischaemia reperfusion injury.

As used herein, the term “C₁-Cₙ alkyl” refers to straight chain and branched saturated hydrocarbon groups generally having from 1 to n carbon atoms. Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, and the like.

As used herein, the terms “drug”, “drug substance”, “active pharmaceutical ingredient”, and the like, refer to a compound that may be used for treating a subject in need of treatment.

As used herein, the term “excipient” refers to any substance that may influence the bioavailability of a drug, but is otherwise pharmacologically inactive.

As used herein, the term “pharmaceutically acceptable” refers to species which are within the scope of sound medical judgment suitable for use in contact with the tissues of subjects without undue toxicity, irritation, allergic response, and the like, commensurate with a reasonable benefit-to-risk ratio, and effective for their intended use.

As used herein, the term “pharmaceutical composition” refers to the combination of one or more drug substances and one or more excipients.

As used herein, the term “subject” as used herein refers to a human or non-human mammal.

Examples of non-human mammals include livestock animals such as sheep, horses, cows, pigs, goats, rabbits and deer; and companion animals such as cats, dogs, rodents, and horses.

As used herein, the term “body” as used herein refers to the body of a subject as defined above.

As used herein, the term “therapeutically effective amount” of a drug refers to the quantity of the drug or composition that is effective in treating a subject and thus producing the desired therapeutic, ameliorative, inhibitory or preventative effect. The therapeutically effective amount may depend on the weight and age of the subject and the route of administration, among other things.

As used herein, the term “treating” refers to reversing, alleviating, inhibiting the progress of, or preventing a disorder, disease or condition to which such term applies, or to reversing, alleviating, inhibiting the progress of, or preventing one or more symptoms of such disorder, disease or condition.

As used herein, the term “treatment” refers to the act of “treating”, as defined above.

As used herein, the term “preventing” refers to a reduction of the risk of acquiring a given disease or disorder, or a reduction in the severity of symptoms of the given disease or disorder if the disease or disorder is acquired after the preventative measure. Hence, “preventing” refers to the prophylactic treatment of a subject in need thereof. The prophylactic treatment can be accomplished by administering an appropriate dose of a therapeutic agent to a subject having a predisposition to a disorder, or at risk of developing a disorder, even though symptoms of the disorder are absent or minimal, thereby substantially averting onset of the disorder, or substantially reducing the severity of symptoms of the disorder if it is acquired after the preventive measure.

As used herein, the term “succinate dehydrogenase inhibitor” or “SDHi” refers to a species that inhibit the action of succinate dehydrogenase.

As used herein, the term “thrombolysis” refers to removal of a blockage to blood flow, for example a blood clot, using chemical means, for example through the use of thrombolytic agents.

As used herein, the term “thrombectomy” refers to removal of a blockage to blood flow, for example a blood clot, using mechanical means.

As used herein, the term “pH lowering” refers to a reduction to normal physiological pH (approximately pH 7.4).

As used herein the term “comprising” means “consisting at least in part of”. When interpreting each statement in this specification that includes the term “comprising”, features other than that or those prefaced by the term may also be present. Related terms such as “comprise” and “comprises” are to be interpreted in the same manner.

### EXAMPLES

### Example 1

C2C12 cells were incubated with DSM (0, 1 or 5 mM) for 15 min at pH 6, 7.4 and 8 before measuring intracellular malonate (FIG. 1) by LC-MS/MS (data presented as mean±S.E.M, n=4 biological replicates, statistical significance assessed by two way-ANOVA with Bonferroni's correction for multiple comparisons). The intracellular delivery of malonate is greatly enhanced in cells at a lower pH.

### Example 2

Malonate levels in murine isolated Langendorff-perfused hearts treated with 5 mM DSM infused at either pH 7.4 or 6 were determined (FIG. 2—data presented as mean±S.E.M, n=4 biological replicates, statistical significance assessed by unpaired, two-tailed Student's t-test). Malonate uptake is shown to be greatly enhanced when infused at a low pH.

### Example 3

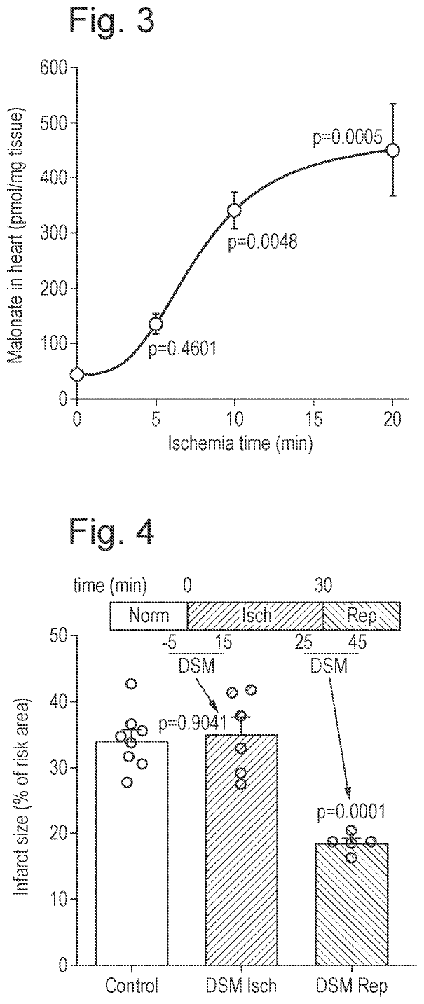

Langendorff-perfused murine hearts were held ischemic for either 0, 5, 10 or 20 min and reperfused with 5 mM DSM (pH 7.4) before malonate levels measured in the heart by LC-MS/MS (FIG. 3—data presented as mean±S.E.M, n=4-6 biological replicates, statistical significance assessed by one way-ANOVA with Bonferroni's correction for multiple comparisons). It is shown that there is an ischemic time-dependent uptake of malonate.

### Example 4

Infarct size was measured in murine LAD model with infusion of DSM (160 mg/kg) at reperfusion quantified by TTC staining (FIG. 4—data presented as mean±S.E.M, n=5-8 biological replicates, statistical significance assessed by one way-ANOVA with Bonferroni's correction for multiple comparisons). Thus, malonate was confirmed to be cardioprotective when administered at the point of reperfusion after ischaemia.

### Example 5

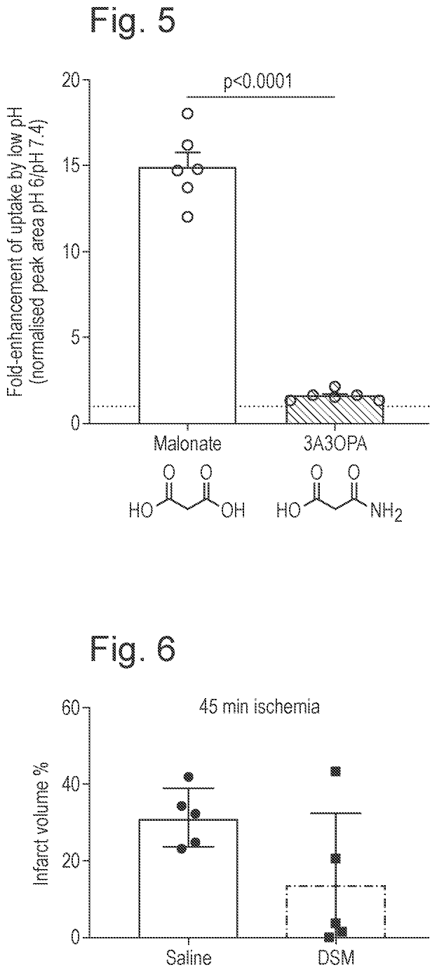

C2C12 cells were incubated with either DSM or malonamate (3-amino-3-oxypropanoic acid—3A3OPA) (both 5 mM) at pH 6 or 7.4 and the intracellular levels measured by LC-MS/MS (FIG. 5—data presented as mean±S.E.M. of the fold-enhancement of uptake at pH 6 versus pH 7.4, n=6 biological replicates, statistical significance assessed by one way-ANOVA with Bonferroni's correction for multiple comparisons). 3A3OPA was unable to react in the same manner as malonate to a lowered pH, with similar levels of uptake achieved for 3A3OPA at both low and neutral pH, demonstrating that low pH is key in driving the uptake of malonate across the membrane.

Malonate has pKa values of 2.83 and 5.69, so at pH 6.4, approximately 16% of the malonate would be in its monocarboxylate form. In 3A3OPA, one carboxylic acid has been replaced with a neutral amido group leaving a single carboxylic acid of pKa˜4.75. Thus, at pH 7.4 3A3OPA resembles the monocarboxylate form of malonate. This led to more 3A3OPA being taken up into cells at pH 7.4 than malonate. Lowering the pH to 6 led to an ˜15-fold increase in malonate uptake while 3A3OPA uptake changed negligibly, because it will remain as a monocarboxylate across this pH range. Together, these data support transport of the monoanionic form of malonate, under the conditions that occur during early reperfusion.

### Example 6

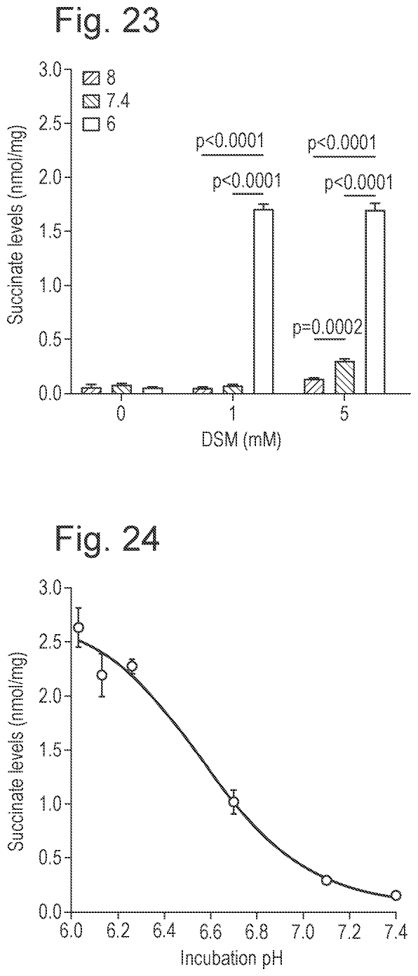

H9c2 myoblasts were incubated with DSM (0, 1 or 5 mM) for 15 min at either pH 6, 7.4 or 8 before measuring intracellular malonate (FIG. 22) and succinate (FIG. 23) by LC-MS/MS (data presented as mean±S.E.M, n=4 biological replicates, statistical significance assessed by two way-ANOVA with Bonferroni's correction for multiple comparisons). FIG. 24 shows succinate levels in C2C12 cells incubated with DSM (5 mM) for 15 min at various pH (data presented as mean±S.E.M, n=3 biological replicates).

At pH 6, the levels of malonate in the cell were significantly higher than at pH 7.4 or 8, thus malonate uptake is favoured by acidic pH. Succinate levels mirrored those of malonate, with greater succinate accumulation as a result of increased malonate-dependent SDH inhibition at low pH. Low pH alone had no effect on succinate levels (FIG. 23), suggesting that the elevated succinate levels are a result of malonate entry into the cells followed by SDH inhibition.

### Example 7

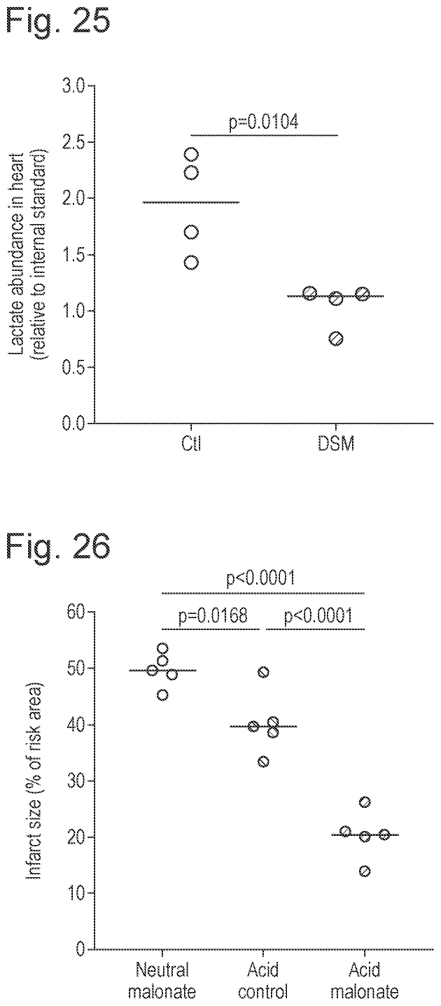

Lactate levels in the Langendorff heart were measured after 20 min ischemia and 1 min reperfusion with and without 5 mM DSM (data presented as mean, n=4 biological replicates, statistical significance assessed by two-tailed, unpaired Student's t-test). Results are shown in FIG. 25. Compared to reperfusion alone, lactate levels decreased in the malonate-treated hearts.

### Example 8

Infarct size was measured in murine LAD MI model with 100 μl bolus of 8 mg/kg DSM, pH 4 acid control or 8 mg/kg pH 4 formulated malonate at reperfusion after 30 min ischemia, quantified by TTC and Evans blue staining after 2 hr reperfusion (data presented as mean, n=5 biological replicates, statistical significance assessed by one way-ANOVA with Bonferroni's correction for multiple comparisons). Results are shown in FIG. 26. This data demonstrates that when a given dose of malonate was re-formulated at pH 4 (a pH currently used in FDA-approved parenteral formulations) and administered as a bolus, this conferred significant protection that was not due to the low pH alone.

### Example 9

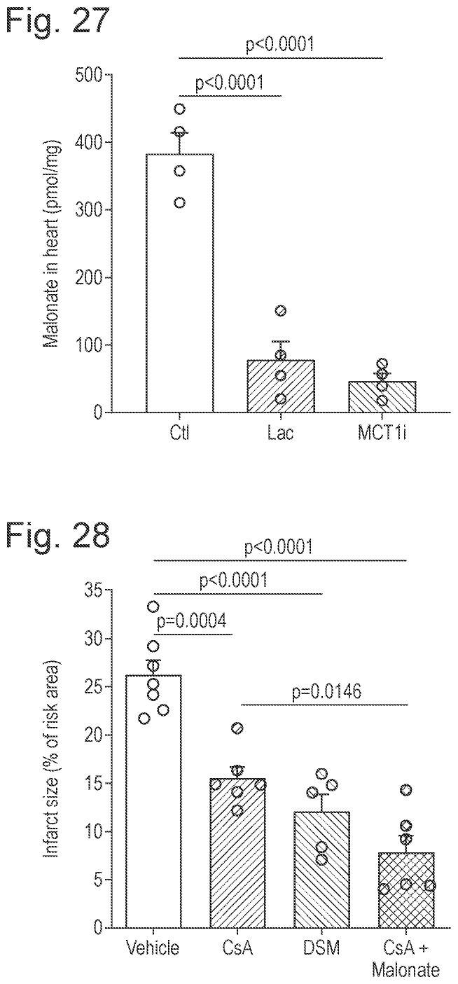

Malonate levels in murine Langendorff hearts perfused at pH 6 for 5 min with 5 mM DSM±lactate (50 mM; Lac) or MCT1 inhibitor (10 μM AR-C141990; MCT1i) (data are mean±S.E.M., n=4, statistical significance assessed by one way-ANOVA with Bonferroni's correction for multiple comparisons). Results are shown in FIG. 27. Malonate uptake into the Langendorff perfused heart was blocked by the MCT1 inhibitor AR-C141990, in the perfusion medium, and by excess lactate. This suggests that MCT1 is responsible for the low pH uptake of malonate. It is noted that MCT1 is also a lactate transporter, so this data also suggests that, at high concentrations, lactate competes with malonate.

### Example 10

Infarct size was measured in murine LAD model with infusion of vehicle (ethanol/Cremphor EL in saline)±cyclosporin A (10 mg/kg) or DSM (160 mg/kg) at reperfusion (data presented as mean±S.E.M, n=5-7 biological replicates, statistical significance assessed by one way-ANOVA with Bonferroni's correction for multiple comparisons). Cyclosporin A is a known inhibitor of the mitochondrial permeability transition pore (PTP). Results are shown in FIG. 28. Cardioprotection from malonate was additive to cyclosporin A alone.

### Reference Example 11—Transient Middle Cerebral Artery Occlusion (MCAO) Model

Male C57B16J mice, 6-8 weeks old, were anesthetized with 3% isoflurane and anaesthesia was maintained on 1.5-2% isoflurane. A Doppler flowmetry probe (Perimed, Sweden) was attached to the skull to continuously monitor the cerebral blood flow. Left common carotid artery (CCA) and left external carotid artery were exposed and permanently ligated. Left internal common carotid artery was clamped temporarily. Next, an incision was made on CCA and a filament with a silicon tip (0.22 mm in diameter, 2-3 mm in length, Doccol, USA) was inserted and guided forward into internal carotid artery until it reached middle cerebral artery (MCA). The occlusion of MCA was confirmed by seeing at least 70% drop in cerebral blood flow. After 45 minutes ischaemia, the filament was retracted to allow reperfusion for 2 hours. The reperfusion was confirmed when the cerebral blood flow reached to 80% of that of baseline.

Either disodium malonate (DSM) or vehicle control was given i.v. for 20 min starting just prior to reperfusion.

At the end of reperfusion, mice were sacrificed by cervical dislocation; brain was collected and sliced at 2 mm thicknesses and stained with 2% triphenyltetrazolium chloride in saline at 37 C for 10-15 min. Then, brain slices were fixed with 4% paraformaldehyde overnight and imaged using a scanner. The healthy tissue area (red-stained) in both hemispheres was measured using ImageJ. The infarct volume % was calculated by first calculating the volume of healthy tissue in each hemisphere (Σ area of each slice×thickness of slice), then by the formula: ((volume of healthy tissue in uninjured hemisphere−volume of healthy tissue in injured hemisphere)÷volume of healthy tissue in uninjured hemisphere)×100.

As shown in FIG. 6, necrotic brain infarct volume was decreased when DSM was given for 20 minutes starting just prior to reperfusion.

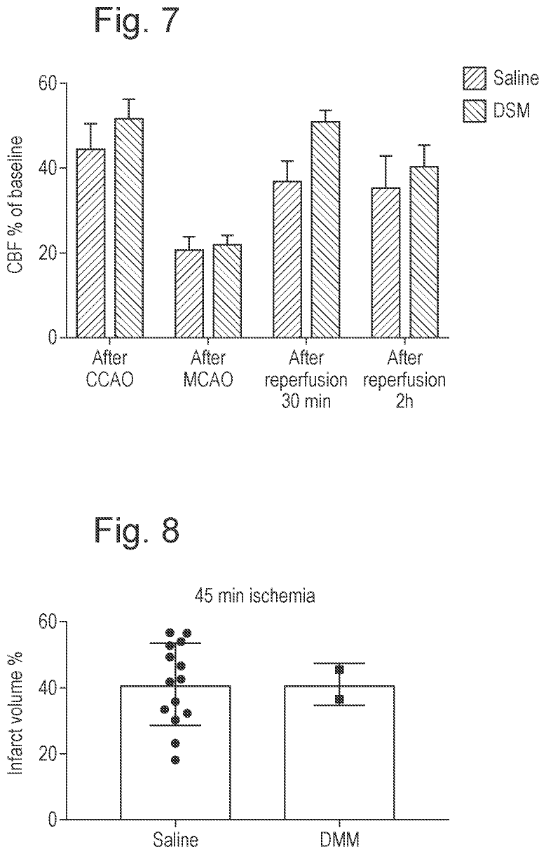

As shown in FIG. 7, cerebral blood flow was significantly reduced during transient middle cerebral artery occlusion (MCAO). Administering DSM for 20 minutes starting immediately prior to reperfusion did not significantly alter the blood flow during the procedure, showing that DSM targets reperfusion injury rather than cerebral blood flow.

### Comparative Reference Example 11

The method of Reference Example 11 was performed, except that dimethyl malonate (DMM) was administered instead of DSM. As shown in FIG. 8, there was no significant difference in necrotic brain infarct volume when DMM was given for 20 minutes starting just prior to reperfusion.

### Reference Example 12—Malonate Uptake In Vitro

We first explored whether malonate could be taken up by cells in culture. C2C12 mouse myoblast cells were plated at 300,000 cells/well and adhered overnight. The next day, cells were treated with either DSM (a—0.25 mM; b—0.25, 1 or 5 mM) or untreated for 0 to 240 min before cooling plates on ice and rapidly washing 4 times with ice-cold PBS and placed on dry ice. Cells were extracted for mass spectrometry with 500 μl extraction buffer (50% methanol, 30% acetonitrile and 20% water) with 1 nmol ¹³C-malonate internal standard centrifuged to remove insoluble debris.

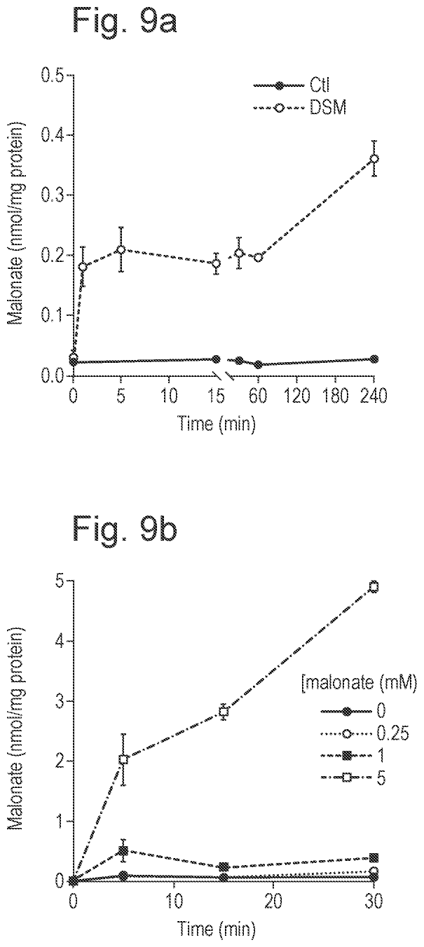

This showed that there was rapid malonate uptake into cells, reaching intracellular levels of ˜0.2-0.4 nmol/mg protein when incubated with 250 μM disodium malonate (FIG. 9a). Malonate uptake was both dose and time-dependent (FIG. 9b), with 5 mM DSM achieving high levels of intracellular malonate (˜2 nmol/mg protein) after 5 min. Allowing for a cell volume of 260 mg protein/ml, the malonate concentration within the cell after 5 min incubated with 250 μM or 5 mM malonate the intracellular concentrations are about 50 and 500 μM, respectively.

### Reference Example 13—Malonate Uptake In Vivo

To see if this uptake also occurred in vivo, we injected disodium malonate into a normoxic mouse by a bolus in the tail vein and measured the malonate levels in tissues 5 min later. C57/BL6 mice were tail vein injected with DSM (160 mg/kg) before cervical dislocation 5 min after the injection, tissues harvested and clamp frozen in liquid nitrogen. For tissue analysis, tissues were weighed and extracted in 25 μl/mg extraction buffer containing internal standard and homogenised in a Precellys 24 homogeniser. Homogenates were centrifuged to remove insoluble debris. Extracts were analysed by LC-MS/MS and the levels of malonate assessed by interpolation of a malonate standard curve.

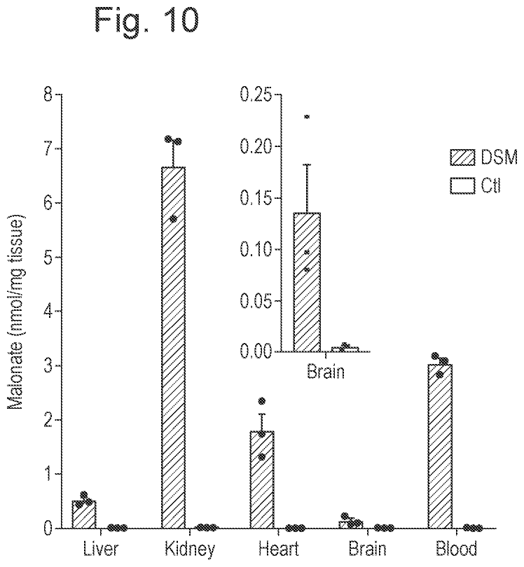

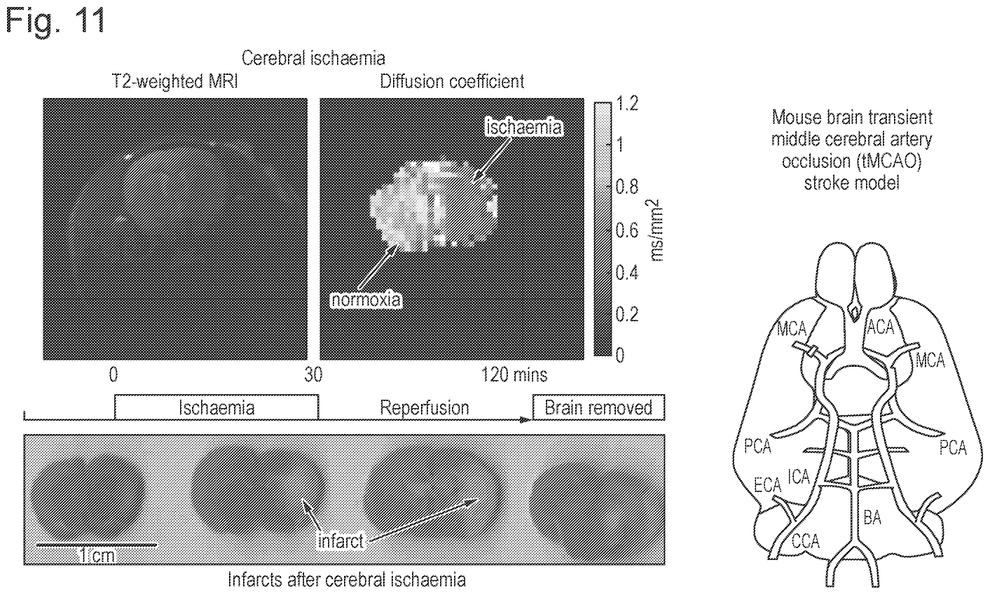

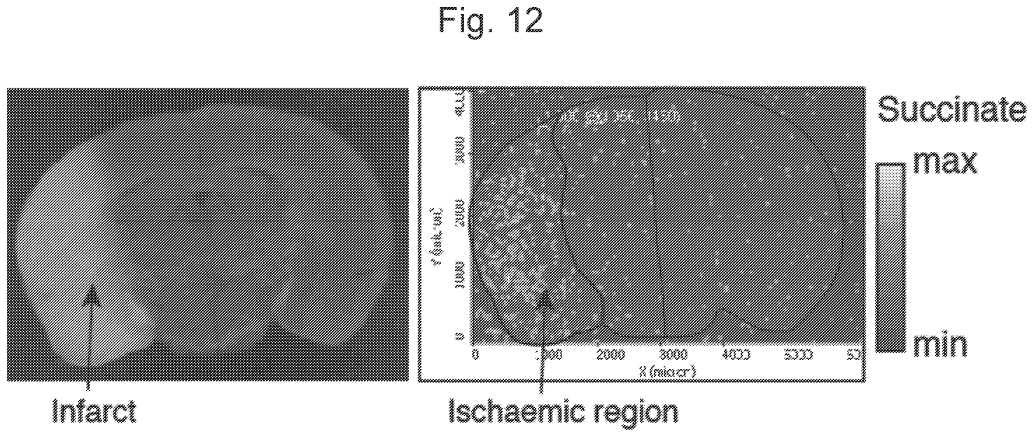

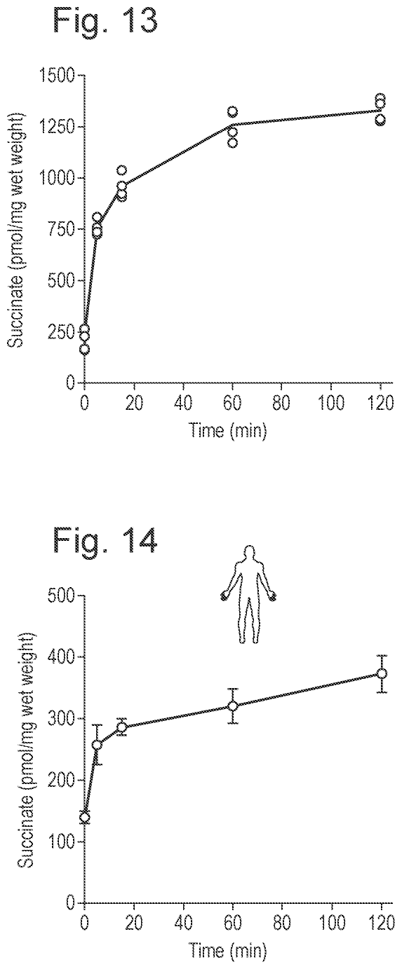

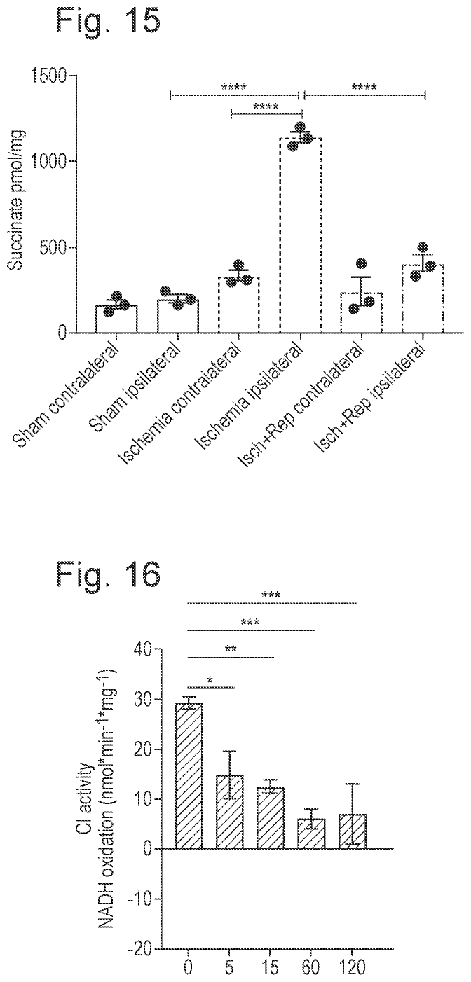

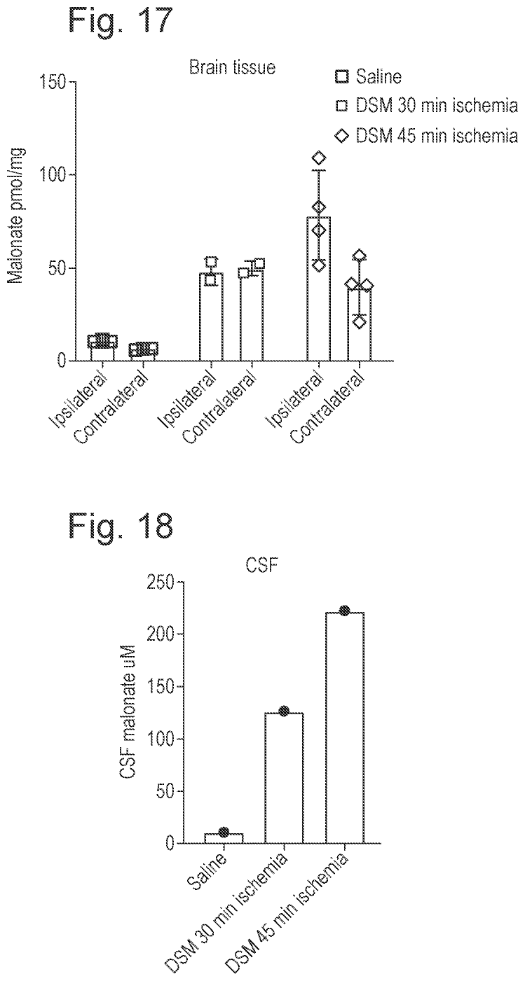

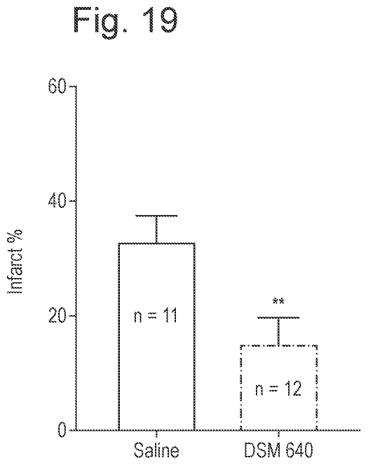

This showed tissue levels of malonate to be particularly high in the kidney with significant amounts in the heart and liver and also uptake in the brain (FIG. 10). This was consistent with tissue uptake of malonate.

### Reference Example 14

### Animals

Male C57B16J mice were purchased from Charles River Laboratories and housed in a room on a 12h light/dark cycle with ad libitum access to food and water. Mice were acclimatized for a week before being used in the experiments. All experimental procedures were carried out in accordance to the UK Animals Act 1986 and the University of Cambridge Animal Welfare policy and were approved by Home Office (Project License 70/8238 and 70/08840).

### Human and Mouse Brain Warm Ischaemia

Mice, 8-10 weeks old were sacrificed by cervical dislocation and the brain were clamp-frozen immediately or after 5, 15, 60 or 120 minutes of warm ischaemia at 37° C. Human brain biopsies were retrieved from patients undergoing surgeries for brain tumours in collaboration with Dr Richard Mair, Neurosurgery Cambridge. The biopsies (20-80 mg) were rapidly cut into 3 to 5 pieces depending on the tissue size. One piece was frozen immediately (time from dissection to freezing: 30 s to 2 min) and the other pieces were incubated at 37° C. for indicated time periods (5 min to 120 min) of warm ischaemia and then clamp-frozen.

### Metabolite Extraction

Approximately 20 mg of mouse brain tissue and 5-20 mg of human brain tissue was weighed on dry ice and placed in pre-chilled Precellys tube (CK28-R, Bertin Instruments, France). Next, 25 μl/mg dry-ice cold extraction buffer (50% [v/v] methanol, 30% [v/v] acetonitrile and 20% [v/v] H₂O), supplemented with 1 nmol of [¹³C₄]-succinate (Sigma Aldrich, UK) was added to the tubes and the tissue was homogenized using a Precellys 24 tissue homogeniser (6,500 rpm, 15 s; Bertin Instruments, France). The homogenizing program was run again after 5 min incubation on dry ice. Samples were then centrifuged at 17000 rpm at 4° C. and the supernatant was collected and incubated in −20° C. freezer for 1h. The centrifuging step was repeated two times and the supernatant was transferred to pre-chilled MS vials and were stored at −80° C. until analysis for succinate by liquid chromatography-tandem mass spectrometry (LC-MS/MS).

### LC-MS/MS for Quantification of Succinate

LC-MS/MS analysis of succinate was performed using an LCMS-8060 mass spectrometer (Shimadzu, UK) with a Nexera X2 UHPLC system (Shimadzu, UK). Samples were stored in a refrigerated autosampler (4° C.) upon injection of 5 μl into a 15 μl flowthrough needle. Separation was achieved using a SeQuant® ZIC®-HILIC column (3.5 μm, 100 Å, 150×2.1 mm, 30° C. column temperature; MerckMillipore, UK) with a ZIC®-HILIC guard column (200 Å, 1×5 mm). A flow rate of 200 μl/min was used with mobile phases of A) 10 mM ammonium bicarbonate and B) 100% acetonitrile. A gradient of 0-0.1 min, 80% MS buffer B; 0.1-4 min, 80%-20% B; 4-10 min, 20% B, 10-11 min, 20%-80% B; 11-15 min, 80% B was used. The mass spectrometer was operated in negative ion mode with multiple reaction monitoring (MRM) and spectra were acquired using Labsolutions software (Shimadzu, UK), with compound quantities calculated from relevant standard curves in MS extraction buffer and comparing against [¹³C₄]-succinate internal standard.

### Measurement of Mitochondrial Complex I Activity

Frozen mouse brain weighing 7-10 mg were lysed in ice-cold 400 ml of 50 mM KH₂PO₄ (KPi buffer) using Precellys CK14 tubes and a Precellys tissue homogenizer (Bertin Instruments) at 6500 rpm, one 15s cycle. The homogenates were rapidly aliquoted and frozen in dry-ice-cold tubes and stored in −80° ° C. until further processing. The protein concentration was measured following standard BCA assay. New aliquots of samples were diluted in KPi buffer containing 0.05% dodecyl maltoside (DDM) to obtain 5 μg total protein amount in 100 μl of buffer for complex I activity assay. In a 96-well plate, 40 μl of the assay buffer (200 μM KCN and 0.3 μM antimycin A in KPi buffer) were added in each well, followed by adding 5 μl of either ethanol+100 μM decylubiquinone or 5 μl of ethanol+0.5 μM of rotenone. The assay was started by adding 50 μl of freshly-prepared 0.8 mM NADH. The NADH oxidation was measured in a plate reader by monitoring absorbance at λ=340 and 380 (8-10 s intervals) for 30 min. The samples were run as duplicates. The maximum linear rate of NADH oxidation was calculated by subtracting the absorbance at 340-380 nm and the background rate was removed by subtracting the rate in samples with rotenone. The NADH concentration was determined using the Beer-Lambert law and the extinction coefficient ε340−380=4.81 mM⁻¹ cm⁻¹.

### Middle Cerebral Artery Occlusion (MCAO) Non-Recovery Model of Stroke

Mice, 8-12 weeks old and weighing 22-30 grams were anesthetized by isoflurane (3% induction, 1.5-2% maintenance) delivered in 100% oxygen. A Doppler (PeriFlux System 5000, Perimed, Sweden) flowmetry probe was attached to the skull on the ipsilateral side to monitor and record the blood circulation at MCA territory. Next, left common carotid artery (CCA) and external carotid artery were exposed and permanently ligated. After temporary occluding the left internal carotid artery by a clamp, an incision was made on CCA, a 6-0 suture with a silicone-coated tip (602223PK10RE, Doccol, USA) was inserted, clamp was removed and the suture was inserted forward until a drop in blood flow was observed on Doppler confirming the MCAO. The ischaemia duration was either 30 min or 45 min and then the suture was removed allowing the reperfusion. For metabolite analysis mice were sacrificed at indicated time points after ischaemia or reperfusion, sacrificed by cervical dislocation and ipsilateral and contralateral regions of the brain were rapidly dissected and clamp-frozen. For infarct measurement, the mice were kept under anaesthesia for a 2h period of reperfusion and then sacrificed by cervical dislocation. The brain was dissected out and sliced freshly for staining and infarct size measurement. Mice were excluded from the study if the drop in blood flow after MCAO was higher than 30% of that of baseline or for surgical reasons such as excessive bleeding. No exclusion was performed after endpoint measurements.

### Matrix-Assisted Laser Desorption/Ionization (MALDI) Imaging of Succinate

Mice underwent MCAO surgery (30 min ischaemia±5 min reperfusion) as above and the brain was immediately dissected out and frozen in liquid nitrogen-cold isopentane. Coronal sections were cut at 20 μm thickness and mounted on a slide (Superfrost Plus, Thermo Scientific, UK) and were rapidly dried on a heat pad at 60° C. and stored in −80° C. until analysis. Two sections per brain at the level of striatum (MCA territory) was used for MALDI. The matrix solution (1,5-diaminonaphtalene in 80:20 MeOH:H2O v/v, 10 mg/ml) was sprayed on the sections (20 layers) using a nebulized sprayer (Suncollect MALDI spotter; KR Analytical, Cheshire, UK). Imaging was performed using a MALDI LTQ Orbitrap XL (Thermo Fisher Scientific, Hemel Hempstead, UK). Spectra were acquired in negative ion mode for mitochondrial metabolites. The identity of metabolites was obtained by comparing observed to theoretical m/z values. Succinate was detected at 117.0166 m/z. Images were reconstructed using ImageQuest (Thermofisher).

Cerebrospinal Fluid (CSF) Collection after MCAO

Mice underwent non-recovery MCAO surgery and were sacrificed by an overdose of sodium pentobarbital at indicated time points. Next, the mice were perfused with ice-cold PBS via left ventricle for 2.5 minute at a rate of 2 ml/min. CSF was collected from cisterna magna using a glass capillary.

### Disodium Malonate Treatment

The non-recovery surgery mice were treated intravenously (i.v.) with disodium malonate (DSM) or the vehicle (phosphate buffered saline, PBS) 10 minutes before reperfusion infused i.v. at a rate of 5 μl/min. Total injected volume was 100 μl. No randomization was performed in non-recovery surgeries, but the investigator measuring the infarct was blinded to the treatment groups. For the recovery surgeries, an investigator randomly ID-numbered Eppendorf tubes containing DSM or PBS. The surgeon randomly chose a tube for treatment. Therefore, the surgeon and investigators analysing the behaviour and infarct volume were blinded to the treatments throughout the experiments and measurements. Mice weighing 22-28 (24.7±1.4; Mean±SD) grams received 4 mg DSM in 100 μl PBS (162.2±9.1 mg/kg; Mean±SD) or the PBS only, infused i.v. at a rate of 10 μl/min starting 5 minutes before reperfusion.

Infarct Volume Measurement after Non-Recovery MCAO

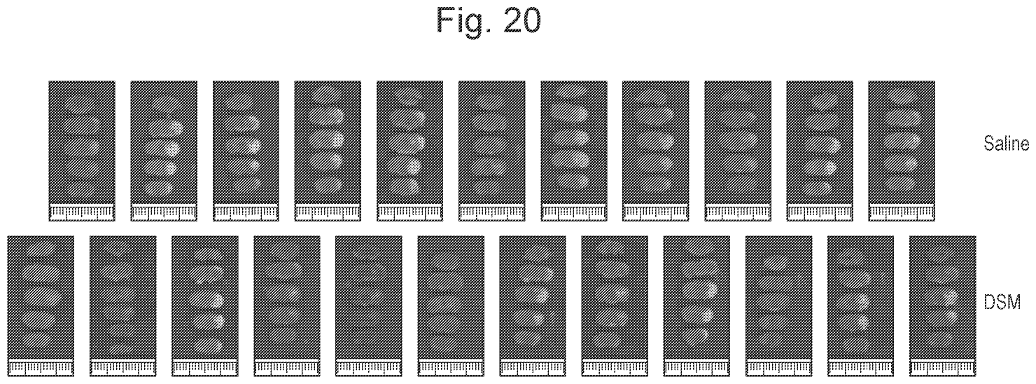

Brains from the mice that underwent non-recovery surgery were immediately sliced at 2 mm thickness and stained in 2% triphenyltetrazolium chloride (TTC) at 37° C. for 10 min to identify infarct area (shown in FIG. 20). The slices were fixed in 4% paraformaldehyde overnight and were imaged using a scanner. The healthy area (stained red) was measured in two hemispheres. The infarct area was calculated as (Contralateral healthy area−ipsilateral healthy area). The infarct volume was calculated as sum of (infarct area×slice thickness) and was presented as percentage of the healthy hemisphere volume.

### Statistics

For infarct size comparing two groups, non-parametric Mann-Whitney test was performed. For multiple group comparisons (blood flow and succinate quantity) one-way or two-way ANOVA was carried out with Tukey's or Sidak's post-hoc tests. All statistical analysis was carried out using Graphpad Prism version 7.0e.

### Reference Example 15—Metabolites in Venous Blood of Stroke Patients

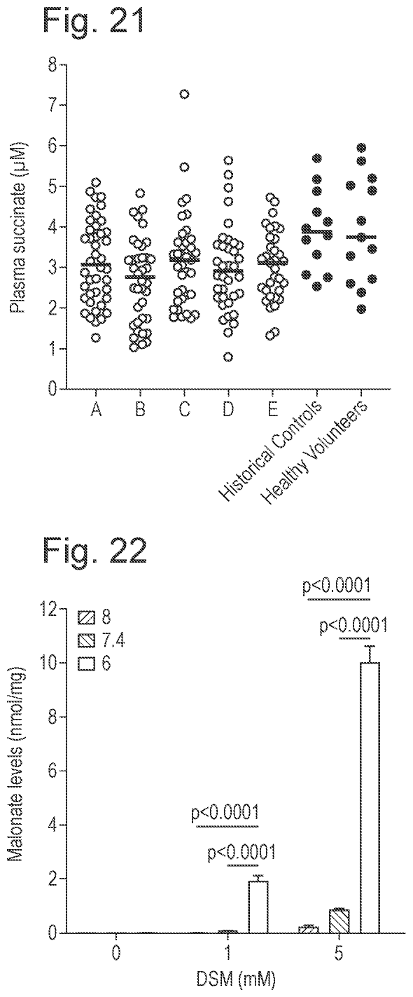

Venous blood was taken from patients before and following thrombolysis due to an acute ischaemic stroke at 5 time points: A—before initiation of thrombolysis; B—5 min, C—15 min, D—30 min, and E—60 min after the start of the thrombolysis. The blood samples were analysed for plasma succinate levels. Healthy controls were age and sex matched. Results are shown in FIG. 21. No increase in succinate was evident in venous blood from patients undergoing thrombolysis.

While specific embodiments of the invention have been described above, it will be appreciated that the invention may be practiced otherwise than as described. The descriptions above are intended to be illustrative, not limiting. Thus it will be apparent to one skilled in the art that modifications may be made to the invention as described without departing from the scope of the claims set out below.

1. A composition comprising a salt of formula (I): 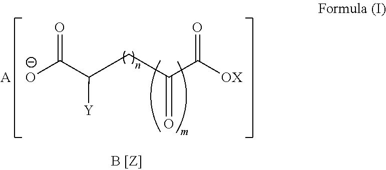
for use in treating or preventing ischaemia reperfusion injury in a subject;
wherein X is selected from a negative charge, H, or C₁-C₁₂ alkyl;
wherein Y is selected from H, OH or C₁-C₁₂ alkyl;
wherein n is 0 or 1;
wherein m is 0 or 1;
wherein Z is one or more pharmaceutically acceptable cations;
wherein A and B are independently any integer, such that the net charge of the salt is 0;
and wherein said composition has a pH of from about 4.0 to about 7.0.

2. The composition for use according to claim 1, wherein said composition has a pH of from about 5.5 to about 6.5.

3. The composition for use according to claim 1 or claim 2, wherein said composition further comprises one or more buffering agents.

4. The composition for use according to claim 3, wherein said one or more buffering agents is selected from citric acid, acetic acid, lactic acid, gluconic acid, aspartic acid, glutamic acid, tartaric acid, succinic acid, malic acid, fumaric acid, carbonic acid, a carbonate salt, a bicarbonate salt, or α-Ketoglutaric acid.

5. The composition for use according to any one of claims 1 to 4, wherein said composition is in the form of a solution for infusion.

6. The composition for use according to any one of claims 1 to 5, wherein said composition further comprises a blood thinning agent and/or a lysis agent.

7. The composition for use according to any one of claims 1 to 6, wherein in said salt of formula (I), n is 0, and m is 0.

8. The composition for use according to any one of claims 1 to 7, wherein said salt of formula (I) is a malonate salt.

9. The composition for use according to any one of claims 1 to 8, further comprising one or more pharmaceutically acceptable excipients, carriers or diluents.

10. The composition for use according to any one of claims 1 to 9, wherein the ischaemia reperfusion injury is stroke ischaemia reperfusion injury.

11. The composition for use according to any one of claims 1 to 9, wherein the ischaemia reperfusion injury is myocardial ischaemia reperfusion injury.

12. The composition for use according to any one of claims 1 to 9, wherein the ischaemia occurs during organ transplantation.

13. The composition for use according to any one of claims 1 to 12, wherein said use comprises administering the composition directly to the tissue undergoing ischaemia.

14. The composition for use according to any one of claims 1 to 13, wherein said use comprises administering said composition to the subject before the onset of reperfusion.

15. The composition for use according to any one of claims 1 to 13, wherein said use comprises administering said composition to the subject at the point of reperfusion.

16. The composition for use according to any one of claims 1 to 15, wherein said use comprises administering said composition as a bolus.

17. The composition for use according to any one of claims 1 to 16, wherein said use comprises administering the composition in combination with a treatment used or intended to remove a blockage in blood flow.

18. The composition for use according to claim 17, wherein the treatment used or intended to remove a blockage in blood flow is selected from thrombolysis by application of blood thinning agents or application of lysis agents, and/or thrombectomy.

19. The composition for use according to claim 17 or claim 18, wherein the treatment used or intended to remove a blockage in blood flow is thrombectomy, and wherein said composition is administered to the ischaemic tissue during the thrombectomy procedure.

20. A combination therapy for use in treating or preventing ischaemia reperfusion injury in a subject, said combination therapy comprising (1) a pH lowering component having a pH of from about 4.0 to about 7.0 and (2) a salt of formula (I) as defined in any one of claim 1, 7 or 8.

21. The combination therapy for use according to claim 20, wherein said pH lowering component comprises one or more buffering agents.

22. A unit dosage form or a fluid bag for intravenous infusion, said unit dosage form or fluid bag for intravenous infusion comprising a composition comprising a salt of formula (I): 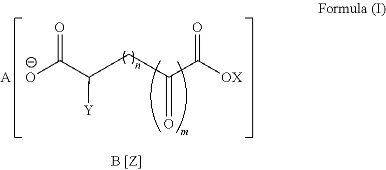
wherein X is selected from a negative charge, H, or C₁-C₁₂ alkyl;
wherein Y is selected from H, OH or C₁-C₁₂ alkyl;
wherein n is 0 or 1;
wherein m is 0 or 1;
wherein Z is one or more pharmaceutically acceptable cations; and
wherein A and B are independently any integer, such that the net charge of the salt is 0;
wherein said composition has a pH of from about 4.0 to about 7.0;
wherein the total volume of the unit dosage form is less than about 20 ml; and
wherein the total volume of the fluid bag for intravenous infusion is less than about 250 ml;
preferably wherein the composition is as defined in any one of claims 2 to 9.

23. A kit comprising (1) a thrombectomy device and (2) a unit dosage form as defined in claim 22 or a composition comprising a salt of formula (I): 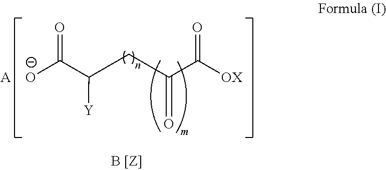
wherein X is selected from a negative charge, H, or C₁-C₁₂ alkyl;
wherein Y is selected from H, OH or C₁-C₁₂ alkyl;
wherein n is 0 or 1;
wherein m is 0 or 1;
wherein Z is one or more pharmaceutically acceptable cations; and
wherein A and B are independently any integer, such that the net charge of the salt is 0; and
wherein said composition has a pH of from about 4.0 to about 7.0;
preferably wherein the composition is as defined in any one of claims 2 to 9.

24. A method of treating or preventing ischaemia reperfusion injury in a subject, the method comprising administering a composition as defined in any one of claims 1 to 9 or a unit dosage form as defined in claim 22 to the subject.

25. Use of a composition as defined in any one of claims 1 to 9 or a unit dosage form as defined in claim 22 for the manufacture of a medicament for treating or preventing ischaemia reperfusion injury.
